(19) United States
(12) Patent Application Publication
(10) Pub. No.: US 2017/0229596 A1
(43) Pub. Date: Aug. 10, 2017

(54) Decorative Composite Body Comprising a Solar Cell

(71) Applicant: D. Swarovski KG, Wattens (AT)

(72) Inventors: Mathias Mair, Völs (AT); Franz Lexer, Axams (AT); Andreas Kurtze, Fritzens (AT)

(21) Appl. No.: 15/316,954

(22) PCT Filed: Oct. 28, 2015
(86) PCT No.: PCT/EP2015/074974

§ 371 Date: Dec. 7, 2016

(30) Foreign Application Priority Data
Oct. 31, 2014 (EP) 14191386.3

(51) Int. Cl. H01L 31/054; A61B 5/11; A44C 9/00; A44C 15/00; A44C 17/00; A44C 5/00

(52) U.S. Cl. CPC H01L 31/0543; H01L 31/0549; A44C 17/008; A44C 5/0007; A44C 9/0053; A44C 15/005; A61B 5/1126; G04C 10/02

(57) ABSTRACT

There is proposed a decorative element containing
(a) a transparent gemstone with a faceted surface comprising convex curved regions,
(b) a wavelength-selective layer, and
(c) a photovoltaic cell.

### FIELD OF THE INVENTION

The invention relates to a decorative element containing a faceted transparent body comprising convex curved regions, a wavelength-selective layer, and a photovoltaic cell. The decorative element is suitable for energy supply, including in the field of wearable electronics.

### BACKGROUND ART

To date, faceted gemstones have been employed almost exclusively for purely aesthetic purposes in accessories and on textiles, but hardly had any functional effect. In the field of wearable electronics (so-called “wearable technologies”), a market with enormous growth opportunities, they are lacking, because this field is associated by the users with functionality rather than decoration. One of the greatest challenges in the field of wearable technologies, such as body sensors, “smart watches” or data glasses, is energy supply, the abrupt failure of which makes the devices inoperative at often unexpected times.

From the patent application US 2013/0329402, energy supply through an incorporated solar cell for decorative elements has been known.

According to the patent specification U.S. Pat. No. 4,173,229, solar cells have also been employed in bracelets and necklaces in order to conduct a therapeutically effective electric current through the body of the jewelry wearers.

The German Utility Model DE 203 03 952 U1 proposes the use of solar cells in “alert locks” for securing jewelry.

EP 2458457 A1 (Casio) relates to a watch in which a transparent member (2), a decorative plate (7), and a solar panel (5) are arranged in this order in a metallic case (1a) from an end side of an opening thereof, wherein the decorative plate has light permeability and has a light refractive part (70) made of a concavo-convex surface portion on the side of the solar panel, and a semi-transmissive reflective plate (6) having both light permeability and light reflectivity is provided between the decorative plate and the solar panel, wherein the semi-transmissive reflective plate has a metal-free vapor deposited film (61). A cross-sectional view shows that the watch has a light-permeable plate on its face that has been ground to be planar from both sides, and has no facets. It is followed by a decorative plate with a concavo-convex surface structure bonded on the bottom thereof to the solar panel. Said concavo-convex layer is a sequence of crests and troughs, i.e., the watch itself has no extensive convex curved regions.

To date, there has been a lack of any technical solution of designing solar cells in a demanding decorative way, so that they may also be processed as gemstones. It is the object of the present invention to adapt a solar cell (photovoltaic cell) in such a way that a highly brilliant decorative element is obtained.

### DESCRIPTION OF THE INVENTION

In a first aspect, the present invention relates to a decorative element containing
(a) a transparent gemstone with a faceted surface comprising convex curved regions,
(b) a wavelength-selective layer, and
(c) a photovoltaic cell.

In a preferred embodiment, elements (a) to (c) are bonded together with adhesive, especially in the mentioned order. In another preferred embodiment, the decorative element consists of elements (a) to (c) bonded together with adhesive, also preferably in that order.

The present invention further relates to the use of the decorative element according to the invention as an energy source, especially in wearable electronic devices. The invention also relates to objects containing a decorative element according to the invention. For example, the decorative element may be advantageously incorporated in so-called “activity trackers”, to which the invention also relates. Further possible applications are mentioned in the following.

Surprisingly, it has been found that a combination of a transparent gemstone with a faceted surface comprising convex curved regions, with a wavelength-selective layer, and a photovoltaic cell is suitable as an energy source for a variety of purposes. According to the invention, the terms photovoltaic cell, photovoltaic (PV) element and solar cell are used interchangeably. The composite bodies according to the invention not only have improved energy supply properties, but they are at the same time gemstones of high brilliancy. Thus, the invention provides energy-supplying functional gemstones, which not only are brilliant, but supply an energy yield that is higher than that of solar cells covered only with a flat glass sheet.

The combination according to the invention provides a variety of possible uses in the design and technology fields, both as an energy source and as a gemstone. In the following, the transparent gemstone with a faceted surface comprising convex curved regions is also referred to as an “optical element”. The decorative elements are highly brilliant and thus enable the use thereof not only as an energy source, but also as a decorative element. The term “transparency” means the ability of matter to transmit electromagnetic waves (transmission). If a material is transparent for incident electromagnetic radiation (photons) of a more or less wide frequency range, the radiation can penetrate the material almost completely, i.e., it is hardly reflected and hardly absorbed. Preferably according to the invention, “transparency” means a transmission of at least 70% of the incident light, preferably more than 80%, more preferably more than 90%. According to the invention, “faceting” means the design of a surface of a gemstone with polygons or so-called n-gons (n>3); facets are usually obtained by grinding a rough crystal, but are also available by pressing methods. The terms “convex” and “concave” relate to an imaginary enveloping area above or below the facets, and the definitions are to be understood by analogy with lenses in optics. The convex and concave regions may be either symmetrical or asymmetrical.

Possible structures of the decorative element (composite body) are shown in FIGS. (1a) to (1c), the reference symbols having the following meanings:
(1) transparent gemstone with a faceted surface comprising convex curved regions;
(2) photovoltaic cell (solar cell);
(3) wavelength-selective coating;
(4) wavelength-selective film;
(5) adhesive;
(6) different structure with respect to the position of the wavelength-selective film;
(7) decorative element.

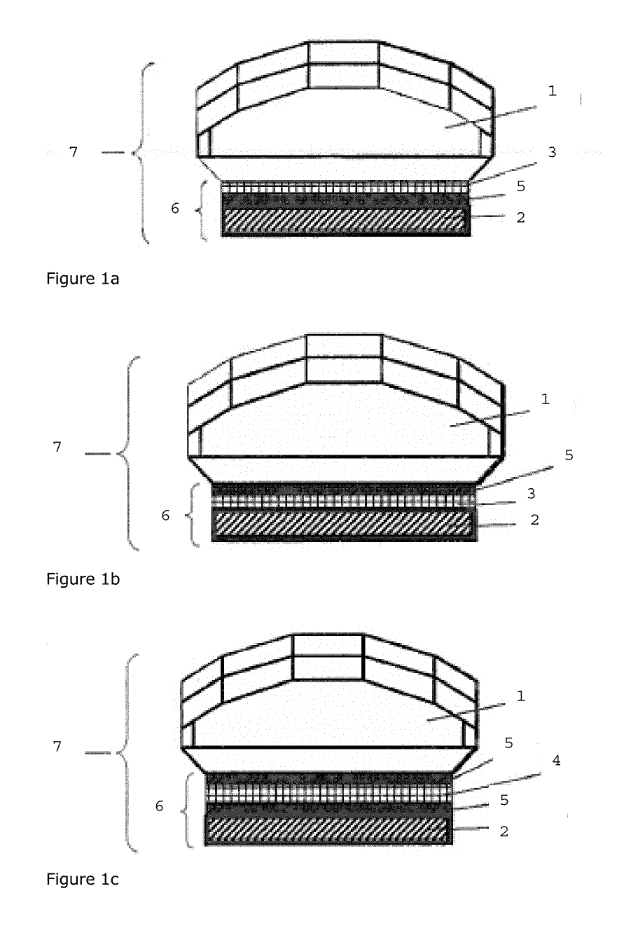

In an embodiment according to the invention, the wavelength-selective coating (see below) may be positioned directly on the planar side opposing the facets, i.e., on the “backside” of the plano-convexo-concave or plano-convex gemstone (FIG. 1a), which is adhesively bonded to the solar cell. In another embodiment according to the invention, the wavelength-selective coating may be positioned on the solar cell, which is adhesively bonded to the gemstone (1) (FIG. 1b). In another embodiment (FIG. 1c), a wavelength-selective film is bonded to the solar cell and to the gemstone (1) by means of two adhesive layers (FIG. 1c). It may be noted that adhesive bonding of the individual parts is not mandatory.

According to the invention, the wavelength-selective layer could also be applied to the faceted surface in principle; however, this is one of the less preferred embodiments because of the possible mechanical abrasion of the layer. The photovoltaic cell may also be prepared by deposition or vapor-deposition of semiconductor materials directly on the optical element, i.e., it need not necessarily be bonded by an adhesive.

The decorative element offers the opportunity to operate various devices in the field of “wearable technologies” in a completely energy self-sufficient way, or else to increase their runtime significantly as a function of the incident light. The permanent recharging of the energy store results in a certain energy level. This allows for a reduction of the charge carrier capacity and consequently of the volume of the energy store. This results in advantages for the design of the product, for example, a more compact design, which may in turn contribute to a reduction of the product cost. Since the complete charging and decharging of a conventional energy store through an external power supply is omitted, a clearly increased service life of the energy stores currently used in the devices results.

One application of the decorative element is represented, for example, by rings and earrings, in which it serves as a gemstone and at the same time provides the necessary energy for an integrated sensor system including a transmission unit. Such systems may serve for the transcutaneous optical measurement of, for example, lactate, glucose or melatonin in the blood. In connection with a specific thin film battery and highly miniaturized electronics, the decorative element for the first time allows a sensor system to be integrated in a compact piece of jewelry. Thus, for example, a ring equipped with the decorative element according to the invention can be employed for the continuous measurement of particular body functions without a battery change.

Also, the partial charging of mobile devices, such as cell phones, laptops, GPS systems or tablet computers, is possible because of a plurality of decorative elements in serial or parallel connection. When a large number of such decorative elements are applied to clothes and accessories, for example, handbags and rucksacks, mobile devices contained therein can be charged inductively. So-called “energy bracelets” that contain the decorative elements according to the invention and are provided with micro USB plugs can be used as mobile charging stations. The decorative element according to the invention may also provide energy for so-called switchable effects, for example, for a color change of a gemstone or, for example, the display functions of a so-called “smart watch”.

The decorative element or a plurality of decorative elements may be integrated into a bracelet, in order to supply energy to, for example, a smart watch or an activity sensor (activity tracker). A reliable electrical interconnection of the decorative elements can be achieved if the decorative elements are interconnected through specific settings. Energy transfer from the decorative elements to the product piece that requires the energy is possible, for example, through a specific spring bar (mainly for watches) or by pogo pins.

Transparent Gemstone with a Faceted Surface Comprising Convex Curved Regions

The gemstone can be made of a wide variety of materials, for example, transparent glass, plastic, transparent ceramic or transparent gems or semi-precious stones. Faceted transparent gemstones made of glass or plastic are preferred according to the invention, because they are lowest cost and are most readily provided with facets. The use of glass is particularly preferred according to the invention. The gemstones comprise convex curved or convexo-concave curved regions. This means that concave curved regions may also be present in addition to the convex curved regions on the faceted side. The side of the gemstone opposite the faceted side is either planar (preferably) or else concave. Gemstones with a plano-convex or plano-convexo-concave geometry are preferred according to the invention, because they enable the most cost-efficient application of crystalline solar cells. Particularly preferred are gemstones of convex, especially plano-convex, geometry.

### Glass

The invention is not limited in principle with respect to the composition of the glass, as long as it is transparent (see above). “Glass” means a frozen supercooled liquid that forms an amorphous solid. According to the invention, both oxidic glasses and chalcogenide glasses, metallic glasses or non-metallic glasses can be employed. Oxynitride glasses may also be suitable. The glasses may be one-component (e.g., quartz glass) or two-component (e.g., alkali borate glass) or multicomponent (soda lime glass) glasses. The glass can be prepared by melting, by sol-gel processes, or by shock waves. The methods are known to the skilled person. Inorganic glasses, especially oxidic glasses, are preferred according to the invention. These include silicate glasses, borate glasses or phosphate glasses. Lead-free glasses are particularly preferred.

For the preparation of the faceted transparent gemstones, silica glasses are preferred. Silica glasses have in common that their network is mainly formed by silicon dioxide (SiO₂). By adding further oxides, such as alumina or various alkali oxides, alumosilicate or alkali silicate glasses are formed. If phosphorus pentoxide or boron trioxide are the main network formers of a glass, it is referred to as a phosphate or borate glass, respectively, whose properties can also be adjusted by adding further oxides. These glasses can also be employed according to the invention. The mentioned glasses mainly consist of oxides, which is why they are generically referred to as oxidic glasses.

In a preferred embodiment according to the invention, the glass composition contains the following components:

(a) about 35 to about 85% by weight SiO₂;
(b) 0 to about 20% by weight K₂O;
(c) 0 to about 20% by weight Na₂O;
(d) 0 to about 5% by weight Li₂O;
(e) 0 to about 13% by weight ZnO;
(f) 0 to about 11% by weight CaO;
(g) 0 to about 7% by weight MgO;
(h) 0 to about 10% by weight BaO;
(i) 0 to about 4% by weight Al₂O₃;
(j) 0 to about 5% by weight ZrO₂;
(k) 0 to about 6% by weight B₂O₃;
(l) 0 to about 3% by weight F;
(m) 0 to about 2.5% by weight Cl.

All stated amounts are to be understood as giving a total sum of 100% by weight, optionally together with further components. The faceting of the gemstones is usually obtained by grinding and polishing techniques that are adequately familiar to the skilled person.

For example, a lead-free glass, especially the glass used by the company Swarovski for Chessboard Flat Backs (catalogue No. 2493), which shows a transmission of >95% in the range of 380-1200 nm, is suitable according to the invention.

### Plastic

As another raw material for the preparation of the faceted transparent gemstone (a), transparent plastics can be employed. All plastics that are transparent after the curing of the monomers are suitable according to the invention; these are adequately familiar to the skilled person. Among others, the following materials are used:
acrylic glass (polymethyl methacrylates, PMMA),
polycarbonate (PC),
polyvinyl chloride (PVC),
polystyrene (PS),
polyphenylene ether (PPO),
polyethylene (PE),
poly-N-methylmethacrylimide (PMMI).

The advantages of the transparent plastics over glass reside, in particular, in the lower specific weight, which is only about half that of glass. Other material properties may also be selectively adjusted. In addition, plastics are often more readily processed as compared to glass. Drawbacks include the low modulus of elasticity and the low surface hardness as well as the massive drop in strength at temperatures from about 70° C., as compared to glass. A preferred plastic according to the invention is poly-N-methylmethacrylimide, which is sold, for example, by Evonik under the name Pleximid® TT70. Pleximid® TT70 has a refractive index of 1.54, and a transmittance of 91% as measured according to ISO 13468-2 using D65 standard light.

### Geometry

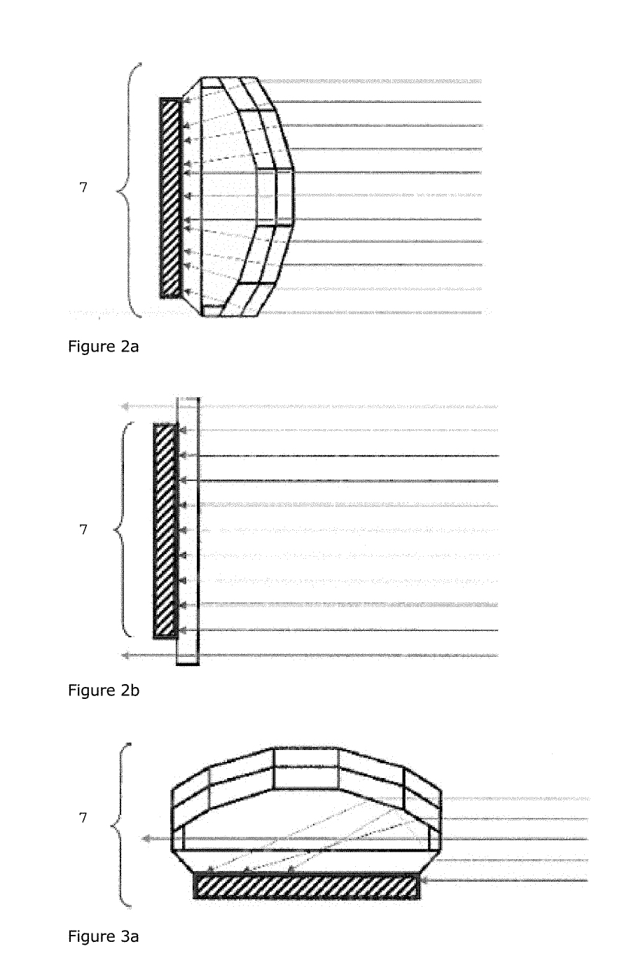

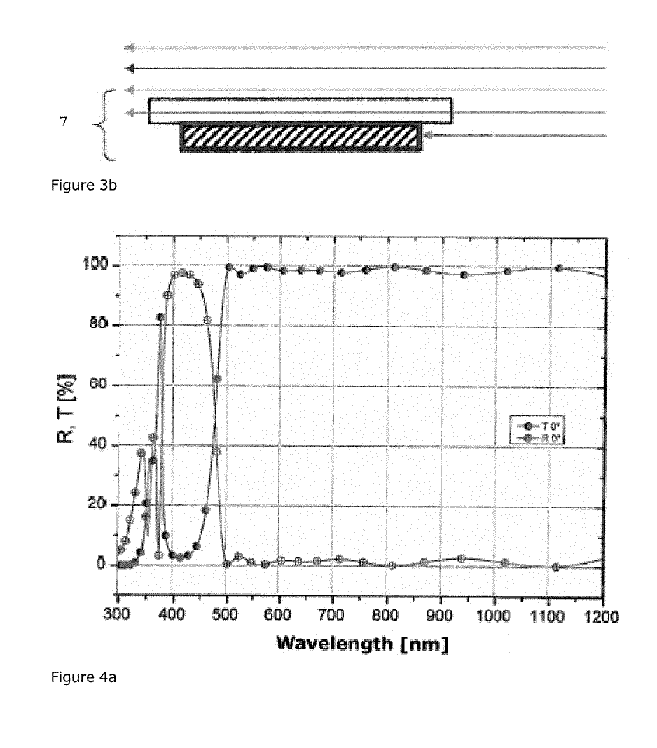

The geometric design of the faceted transparent gemstone is not limited in principle and predominantly depends on design aspects. The gemstone is preferably square, rectangular or round. The faceted transparent gemstone preferably has a convex, especially a plano-convex geometry (cf. FIG. 2a). Preferably, the gemstone contains a plurality of facets on the preferably convex-curved side; preferred are rectangular, especially square, facets, because these contribute to the optimization of the energy yield. The geometry of the gemstone with convex and optionally additional concave regions increases the light yield by increasing the overall surface. While the wavelength-selective layer (see below) has a negative effect on the light yield because some part of the incident light is reflected, this loss is more than compensated by the specific geometry with convex and optionally concave curved regions in combination with the facets. In particular, the convex geometry of the gemstone contributes to a critical reduction of the angular dependence of the energy yield of the solar cell. Especially in view of wearable electronics, in which orientation towards the light source is hardly possible, reduction of the angular dependence is of very great importance. The combination of convexity and faceting (FIG. 2a) focuses the light beams on the surface of the photovoltaic element, and increases the energy yield significantly as compared to a planar geometry (FIG. 2b). At the same time, as shown in FIG. 3a, the angular dependence is dramatically reduced as compared to a thin plate as usually used for encapsulating solar cells (FIG. 3b). Because of the convex curvature in combination with the faceting and the additional area resulting therefrom, the light beams incident on the decorative element are refracted towards the normal onto the solar cell. The faceting results in a multiple reflection of the light beams (light trapping) and thus in an increase of the light yield.

In a preferred embodiment according to the invention, the surface proportion of the convex region is at most ⅓ of the total faceted surface of the gemstone. In this case, the light yield of a convexo-concave geometry is similar to that of an exclusively convex geometry. This could be shown by simulations (see below).

The type of faceting is closely related to the geometry of the optical element. In principle, the geometric shape of the facets is not limited. Preferred according to the invention are square or rectangular facets, especially in combination with a transparent gemstone with square or rectangular dimensions and a plano-convex geometry. However, faceted gemstones that are round may also be used.

### Wavelength-Selective Layer

The wavelength-selective layer enables the decorative element to be used as a gemstone at all. The decorative element obtains a brilliant appearance therefrom. The wavelength-selective layer is preferably provided between the transparent faceted gemstone comprising convex curved regions and the photovoltaic element. Preferably according to the invention, it will be realized in two different ways: by a wavelength-selective film or a wavelength-selective coating, which is prepared by PVD, CVD or wet-chemical methods. However, a wavelength-selective layer may also be obtained from a microstructured surface. The methods of microstructuring are well known to the skilled person.

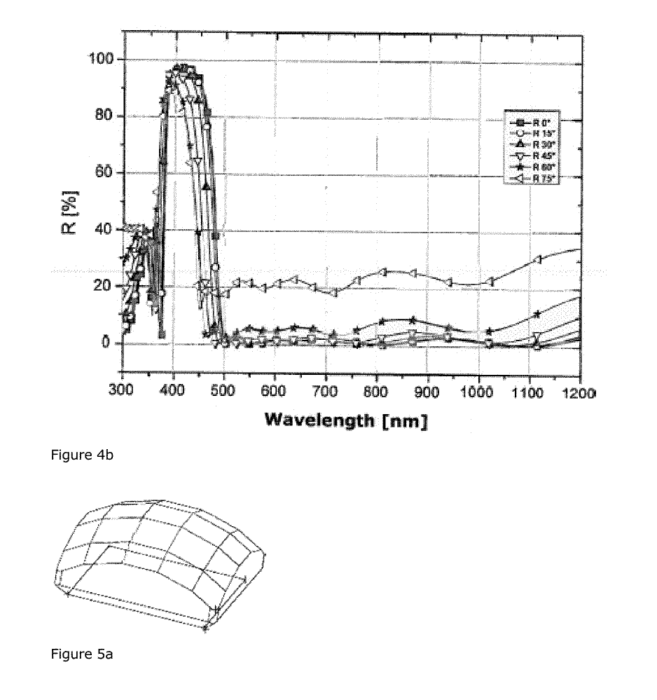

As a result of the reflection of a defined range (=filtering) of the visible spectrum, the optical element gains brilliance and appears in a particular color to the viewer. The brilliance is additionally supported by the faceting of the gemstone. In a preferred embodiment of the invention, the wavelength-selective layer reflects a fraction of the light in the range of 380 to 850 nm, i.e., predominantly in the visible range. The fraction of the light that is reflected is within as narrow as possible a range of the visible spectrum, typically in a range with a width of no more than 50 to 250 nm. On the one hand, this fraction is sufficient to conceive the decorative element as a gemstone with respect to brilliancy. On the other hand, losses in energy yield resulting from the reflected wavelength range are minimized. Therefore, it is preferred according to the invention that the wavelength-selective layer reflects at least 50% of the incident light in a 50 to 250 nm wide reflection interval within a range of from 380 to 850 nm. Preferably, the reflection interval is 50 to 200 nm wide, more preferably 50 to 150 nm. In another preferred embodiment, the wavelength-selective layer has an average transmission of >60%, preferably >80%, outside the reflection interval in a wavelength range of 400 to 1200 nm (cf. FIG. 4a), as measured under an incident angle of the light beams of 0°. Preferably, the wavelength-selective layer is applied to the side of the gemstone opposing the faceted side; alternatively, it may also be applied directly to the photovoltaic element.

The photovoltaic cell (solar cell) can utilize only part of the solar spectrum. The wavelength-selective layer, which acts as a filter, preferably additionally reflects that part of the spectrum that is within the IR range and can no longer be utilized by the solar cell, and thus prevents additional heating of the solar cell.

Usually, solar cells lose 0.47% of energy yield per degree centigrade of heating, so that the correct choice of the coating is of great importance. The shorter the incident wavelength, the higher is the energy of the photons (E=h·ν [eV]). In silicon solar cells, an energy of 1.1 eV is required to strike an electron-hole pair out of the p/n junction; the excess energy is converted to heat. For example, if a photon with 3.1 eV, corresponding to the energy at 400 nm, impinges on the cell, 2 eV is converted to thermal energy, leading to a reduction of the energy yield. Therefore, according to the invention, it is particularly advantageous to reflect the short-wave blue or green fraction (wavelength: 380-490 nm), because the most heat is generated here. In principle, the wavelength-selective layer enables decorative elements with a wide variety of colors to be generated. However, in order to optimize the energy yield, it is preferred that the wavelength-selective layer reflects a fraction from the short-wave range of the visible spectrum.

The wavelength-selective layer shows angle-dependent reflection (FIGS. 4a and 4b). The reflection interval is shifted as a function of the angle of incidence of the light onto the facets. Depending on the position of the facets, different color fractions are reflected to create an almost iridescent effect, i.e., a gradual color change from facet to facet, which cannot be achieved by a plano-convex lens without facets.

In order to enable bonding of the individual components of the decorative element with UV-curing adhesives, it is preferably at least partially transparent to UV light.

Wavelength-Selective Films

Wavelength-selective films are commercially available under the designation “Radiant Light Film”. These are multilayered polymeric films that can be applied to other materials. These optical films are Bragg mirrors and reflect a high proportion of the visible light and produce brilliant color effects. A relief-like microstructure within a range of several hundred nanometers reflects the different wavelengths of the light, and interference phenomena occur, the colors changing as a function of the viewing angle.

Particularly preferred films according to the invention consist of multilayered polymeric films whose outermost layer is a polyester. Such films are sold, for example, by the company 3M under the name Radiant Color Film CM 500 and CM 590. The films have a reflection interval of 590-740 nm or 500-700 nm.

The wavelength-selective film is preferably bonded with the photovoltaic cell and the faceted transparent gemstone by means of an adhesive. The adhesive should also be transparent. In a preferred embodiment, the refractive index of the adhesive deviates by less than ±20% from the refractive index of the faceted transparent body with the convex geometry. In a particular preferred embodiment, the deviation is <10%, even more preferably <5%. This is the only way to ensure that reflection losses because of the different refractive indices can be minimized. The refractive indices can also be matched to one another by roughening the respective boundary layers (moth eye effect). So-called “moth eye surfaces” consist of fine nap structures that change the refraction behavior of the light, not suddenly, but continuously in the ideal case. The sharp boundaries between the different refractive indices are removed thereby, so that the transition is almost fluent, and the light can pass through unhindered. The structural sizes required for this must be smaller than 300 nm. Moth eye effects ensure that the reflection at the boundary layers is minimized, and thus a higher light yield is achieved in the passage through the boundary layers.

Adhesives that can be cured by means of UV radiation are preferred according to the invention. Both the UV-curing adhesives and the methods for determining the refractive index are well known to the skilled person. Particularly preferred according to the invention is the use of acrylate adhesives, especially of modified urethane acrylate adhesives. These are sold by numerous companies, for example, by Delo under the designation Delo-Photobond® PB 437, an adhesive that can be cured by UV light within a range of 320-42 nm.

### Wavelength-Selective Coating

The coating materials are well known to the skilled person. In a preferred embodiment of the invention, the wavelength-selective coatings contain at least one metal and/or metal compound, such as metal oxides, metal nitrides, metal fluorides, metal carbides or any combination of such compounds in any order, which are applied to the faceted gemstones by one of the common coating methods. Successive layers of different metals or metal compounds can also be applied. The methods of preparing coatings and the coatings themselves are adequately known to the skilled person. These include, among others, PVD (physical vapor deposition) methods, CVD (chemical vapor deposition) methods, paint-coating methods and wet chemical methods according to the prior art. PVD methods are preferred according to the invention.

The PVD methods are a group of vacuum-based coating methods or thin-layer technologies, which are adequately familiar to the skilled person and are employed in the optical and jewelry industries, in particular, for coating glass and plastics. In a PVD process, the coating material is transferred to the gas phase. The gaseous material is subsequently passed to the substrate to be coated, where it condenses and forms the target layer. With some of these PVD methods (magnetron sputtering, laser beam evaporation, thermal evaporation etc.), very low process temperatures can be realized. A wide variety of metals can be deposited in this way in a very pure form in thin layers. If the process is performed in the presence of reactive gases, such as oxygen, then metal oxides may also be deposited. A preferred method according to the invention is a coating process by means of sputtering. A typical layer system may be constituted of only one, but also of a large number of layers, depending on the requirement for the function and optical appearance. In practice, the number of layers is mostly limited to between 1 and 25. The typical layer thickness varies between 5 and 800 nm. According to the invention, suitable coating materials include, in particular, Cr, Cr₂O₃, Ni, NiCr, Fe, Fe₂O₃, Al, Al₂O₃, Au, SiOₓ, Mn, Si, Si₃N₄, TiOₓ, Cu, Ag, Ti, CeF₃, MgF₂, Nb₂O₅, Ta₂O₅, SnO₂, ZnO₂, MgO, CeO₂, WO₃, Pr₂O₃, Y₂O₃; BaF₂, CaF₂, LaF₃, NdF₃, YF₃; ZrO₂, HfO₂, ZnS, oxynitrides of Al, Si, and SnZnO.

In order to obtain a wavelength-selective coating, for example, absorbing materials may be used that transmit or reflect only certain proportions of the visible light in a wavelength-selective way because of their absorption behavior, and are therefore colored. Preferably suitable according to the invention are layer systems, constituted by dielectric materials, that transmit or reflect only particular fractions of the visible light because of interference phenomena, and thereby appear colored, for example, a multiple sequence of TiO₂ and SiO₂. A particularly preferred wavelength-selective coating according to the invention consists of an alternate sequence of TiO₂ and SiO₂ in twelve layers and layer thicknesses that vary between about 20 and 145 nm. Preferred according to the invention are so-called band-stop filters with the edge positions 380 and 480 nm, i.e., that the major part of the light is reflected within a range of 380-480 nm (=reflection interval; cf. FIG. 4a). For preparing band-stop filters of other edge positions, the number and thickness of the layers are varied. A variety of commercially available machines are available for PVD layer production, for example, the model BAK1101 of the company Evatek.

### Photovoltaic Element

The photovoltaic element (solar cell) is an electrical component that converts short-wave radiation energy, usually sunlight, directly to electric energy. Which kind of solar cell is employable depends on the required energy supply and the specific application purpose. For the application purpose according to the invention, inorganic solar cells, in particular, are suitable. They are fabricated from semiconductor materials, most commonly silicon. In addition, cadmium telluride, copper indium gallium diselenide and gallium arsenide are employed, inter alia. In so-called tandem solar cells, layers of different semiconductors are used, for example, indium gallium arsenide in combination with indium gallium phosphide.

In addition to the material, the structure of the solar cell is of importance. For example, stacking techniques with combinations of materials are used to increase the efficiency of the overall assembly. The materials are selected in such a way that the incident solar spectrum is utilized maximally. While the theoretically obtainable efficiency is about 43%, only about 15 to 20% is achieved in standard solar cells in reality. Losses arise from recombinations of the charge carriers with accompanying heat generation, from reflection and because of the serial resistance. The voltage at maximum power (maximum power point, MPP) is about 0.5 V for the commonest cells (crystalline silicon cells).

In recent years, the structure of solar cells has been optimized, so that as much light as possible is absorbed, and as much free charge carriers as possible are generated in the active layer. Thus, an anti-reflective layer is applied to the upper side of the solar cell, while the backside is mirrored. The anti-reflective layer provides for the typical bluish to black color of solar cells. The anti-reflective layer is often prepared from silicon nitride, silicon dioxide and titanium dioxide. The layer thickness of the anti-reflective coating also determines the color (interference color). A uniform layer thickness is important because variations on a nanometer scale already increase the reflectance. A blue reflection results from the adjustment of the anti-reflective layer to the red part of the spectrum, the preferred absorption wavelength of silicon. Silicon nitride and silicon dioxide as materials of the anti-reflective layer additionally serve as a passivation layer, which decreases the recombination of charge carriers at the surface, so that more charge carriers are available for electricity generation. A further increase of efficiency is achieved if the front side contact fingers are attached to the backside of the solar cell. This avoids shading on the front side, which would result in a smaller active area and consequently a lower light yield, because up to 10% of the surface would be covered by the metal contacts. In addition, backside contact fingers can be electrically contacted more easily and with less losses as compared to front-side contact fingers. Backside contacted solar cells are preferred according to the invention. Such so-called IBC (interdigitated back contact) cells are marketed, for example, by the company SunPower. In particular, solar cells of monocrystalline silicon and an anti-reflective coating of silicon nitride are suitable according to the invention; preferably, the solar cells have an efficiency of >20%. Particularly suitable according to the invention is the Sunpower® C60 solar cell made of monocrystalline silicon, which is characterized by an efficiency of about 22.5%. The anti-reflective coating of silicon nitride (Si₃N₄) typically has a refractive index of 1.9-2.5. Backside contacting, backside mirroring, a passivation layer of silicon dioxide and the use of n-doped silicon, inter alia, contribute to the increase of efficiency of the solar cells.

The size/area employable according to the invention of the solar cell and of the decorative element according to the invention depend on the application and on the irradiance. For an area of 1 cm² and a cell efficiency of about 20%, up to 20 mWh of energy can be theoretically collected within an hour in direct sunlight at an irradiance of 100 mW/cm². In practice, this value will be somewhat lower because of electrical losses in the charging of the energy store and the fact that an average irradiance of about 100 mW/cm² or 1000 W/m² is not frequently reached in Central Europe. Based on a commercially available “activity tracker” having an average decharging of about 3 mWh/day, an irradiation time of one hour per week in direct sunlight would be sufficient for an area of 1 cm² of solar cell. Because of the good performance of IBC solar cells even under non-ideal light conditions, the use thereof in interior spaces is sufficient to counteract decharging of the wearable electronic devices. As compared to direct sunlight in the open, the irradiance in rooms is lower by a factor of 100-200. The above mentioned sensors for monitoring body functions show an average decharging of about 1 to 5 mWh/day. Here too, energy supply through the decorative element according to the invention is possible, for example, by integrating the decorative element or a plurality of such elements in decorative designs.

In a preferred embodiment of the invention, the photovoltaic element is provided with electric contacts to conduct the generated electric charge carriers off in the form of electric current. The backside electric contacts of the solar cell are contacted through a circuit board and joined into one positive and one negative contact.

In the following, the invention will be illustrated further by means of Examples and Figures without being limited thereto. The Figures show the following objects:

FIG. 1a: Structure of a decorative element with a wavelength-selective coating on the planar side opposing the faceting.

FIG. 1b: Structure of a decorative element with a wavelength-selective coating on the solar cell.

FIG. 1c: Structure of a decorative element with a wavelength-selective film.

FIG. 2a: Focusing light beams on the solar cell in a plano-convex optical element with faceting.

FIG. 2b: Beam path for a planar covering of the solar cell.

FIG. 3a: Refraction of laterally entering light beams in a plano-convex optical element with faceting.

FIG. 3b: Beam path for laterally entering light beams for a planar covering of the solar cell.

FIG. 4a: Spectrum of the wavelength-selective filter coating according to Table 1; T=transmission; R=reflection.

FIG. 4b: Angular dependence of reflection in the wavelength-selective filter coating; R=reflection.

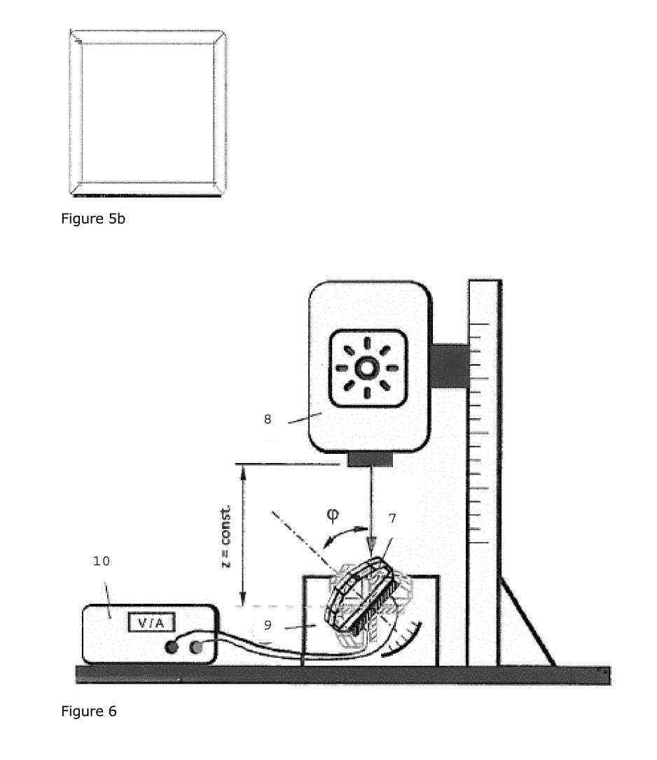

FIG. 5a: Geometry of the optical element; in perspective.

FIG. 5b: Base area of the optical element; 45° chamfer at the base area.

FIG. 6: Measuring set-up—schematically.

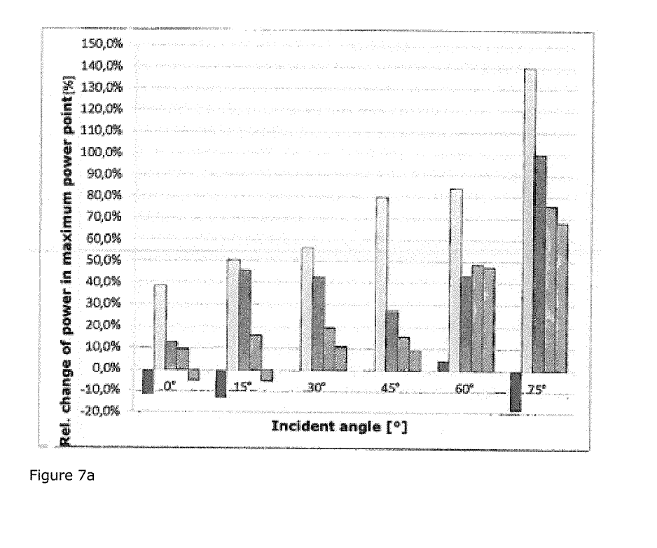

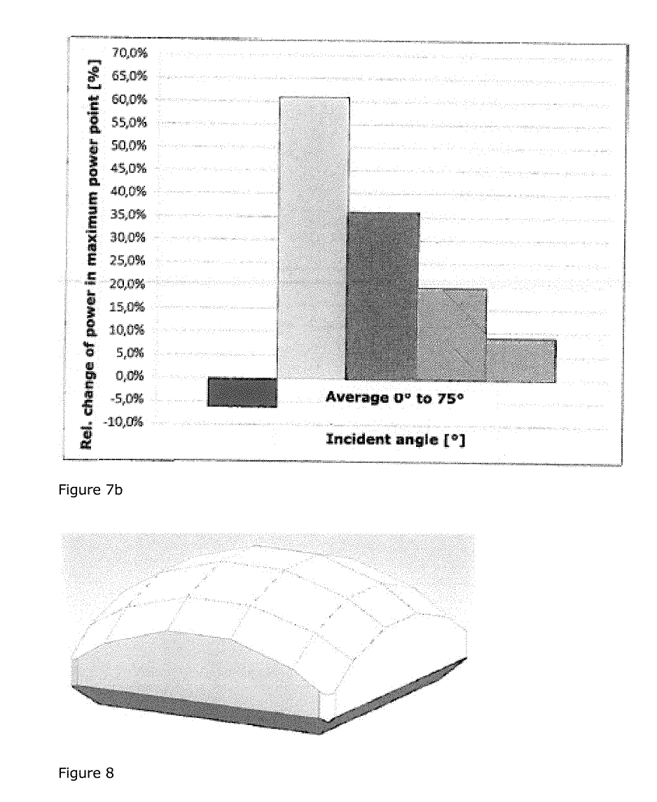

FIG. 7a: Relative change of power at the maximum power point as a function of the angle of incidence of the radiation.

FIG. 7b: Relative change of power at the maximum power point after application of optical elements, averaged over the angles of incidence of 0-75°.

FIG. 8: Geometry of the optical element for the simulation.

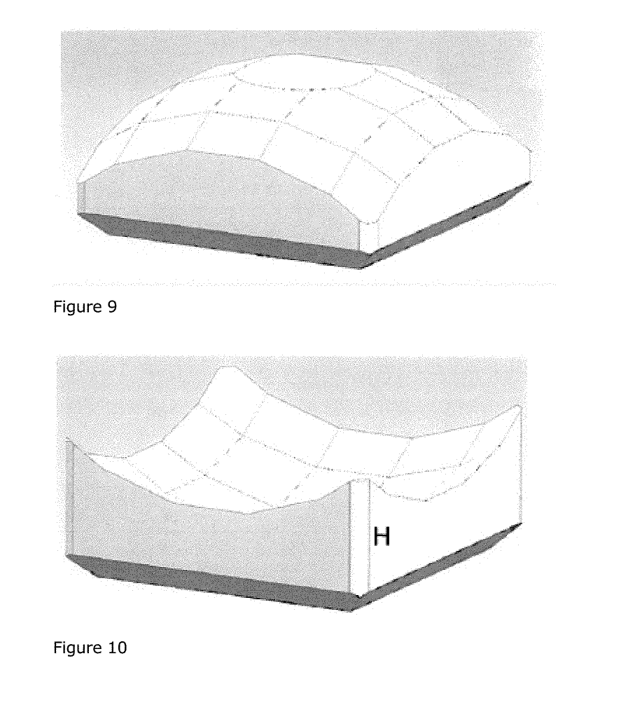

FIG. 9: Geometry of the optical element with plano-convexo-concave curvature.

FIG. 10: Geometry of the optical element with plano-concave curvature.

### INDUSTRIAL APPLICABILITY

The invention further relates to, on the one hand, the use of the decorative element according to the present invention as an energy source, especially in wearable electronic devices, and to objects, especially jewelry, such as rings, necklaces, bracelets and the like, containing at least one decorative element according to the present invention.

### Examples

### Materials

Different decorative elements of different materials and geometries were examined. The decorative elements were assembled from solar cells and optical elements. The Examples according to the invention were additionally provided with a wavelength-selective layer.

Solar Cells.

Solar cells of the type Sunpower C60 (10 mm×10 mm) were used.

Optical Elements—with and without Coating.

The optical elements of glass were produced by methods known to the skilled person from commercially available “Chessboard Flat Back” 2493 elements (30 mm×30 mm) of the company Swarovski.

The optical elements of Pleximid TT70 were produced by plastic injection molding methods in a mold prefabricated for this purpose. For this method, an injection molding machine of the company Engel of the type e-victory 80/50 was used; temperature of barrel: 210° C. increasing to 280° C., nozzle 280° C.; temperature of mold: 180° nozzle side, 140° ejector side; injection pressure limit: 1200 bar; injection speed: about 15 cm³/s; embossing pressure: about 800 bar; no solvents.

Geometry.

The optical elements of Examples 1 and 2 according to the invention and of Comparative Examples C2 and C3 are faceted bodies with 12 mm edge length and a square base area with slightly rounded corners (FIGS. 5a and 5b). A chamfer at an angle of 45° is provided on the base area, so that the actually remaining base area is 10 mm×10 mm (cf. FIGS. 5a and 5b). The faceted upper part with 25 facets in a square arrangement forms a ball segment. The total height of the solid is 5.56 mm, the corner edge height is 1.93 mm.

### DESCRIPTION OF THE EXAMPLES AND COMPARATIVE EXAMPLES

Comparative Example C1: 12 mm×12 mm glass sheet of 0.5 mm thickness; refractive index n=1.52.

Comparative Example C2: An optical element produced from glass according to FIGS. 5a and 5b; dimensions as described above (Geometry); without wavelength-selective coating.

### Example 1

An optical element produced from glass according to FIGS. 5a and 5b; dimensions as described above (Geometry); with the wavelength-selective coating described below.

### Comparative Example C3

An optical element produced from Pleximid TT70 according to FIGS. 5a and 5b with n=1.54; dimensions as described above (Geometry); without wavelength-selective coating (Comparative Example).

### Example 2

An optical element produced from Pleximid TT70 according to FIGS. 5a and 5b; dimensions as described above (Geometry); with the wavelength-selective coating described below.

### Wavelength-Selective Layer

The optical elements according to Examples 1 and 2 were coated in a PVD facility (see above). The structure of the wavelength-selective coating is represented in Table 1:

TABLE 1
Layer structure of the wavelength-selective coating

| N | Material | Physical layer thickness [nm] |
|---|---|---|
| 1 | TiO₂ | 23.9 |
| 2 | SiO₂ | 43.2 |
| 3 | TiO₂ | 64.8 |
| 4 | SiO₂ | 28.7 |
| 5 | TiO₂ | 61.5 |
| 6 | SiO₂ | 33.7 |
| 7 | TiO₂ | 57.7 |
| 8 | SiO₂ | 37.5 |
| 9 | TiO₂ | 66.1 |
| 10 | SiO₂ | 30.5 |
| 11 | TiO₂ | 42.6 |
| 12 | SiO₂ | 141.4 |

### Measuring Set-Up and Measurements

The measurements aimed at examining the influence of the optical element and of the coating on the energy yield of the solar cell as a function of the incident angle of the light beams.

Decorative Elements:

Five different decorative elements assembled from the optical elements according to the Examples and Comparative Examples and the solar cells of the type Sunpower C60 (10 mm×10 mm) were examined.

The measurements were performed with an Oriel Instruments LED sun simulator Verasol-2 (class AAA) using a Keithley 2602A Sourcemeter and a Linos rotary support including the corresponding fixture.

A schematic representation of the measuring set-up is shown in FIG. 6. The reference symbols represent as follows:

(7) decorative element;
(8) sun simulator;
(9) rotary support;
(10) sourcemeter.

Using a sun simulator (8) certified according to class AAA (spectral matching, spatial uniformity, time stability), a constant irradiance of 1000 W/m² was selected for the complete experimental series. The incident angle of the radiation from the light source onto the specimens to be measured according to Examples 1 and 2 as well as Comparative Examples C1 to C3 was varied by means of a rotary support (9), on which the specimens were positioned. The distance z between the center of the solar cell and the sun simulator was kept constant (cf. FIG. 6).

Using a sourcemeter (10), the current-voltage characteristic of each of the solar cells was measured at an irradiance of 1000 W/m², and the power in the maximum power point was determined therefrom.

Each of the five solar cells was measured first without optical elements. The incident angle of the light beams was varied in 15° steps from 0° to 75° (cf. FIG. 6). Subsequently, an optical element (see above) with a wavelength-selective coating (Table 1) was applied to each solar cell using a UV-curable adhesive with a refractive index n=1.461, and the complete measuring series was repeated. Each individual measurement was repeated three times; from this, the arithmetic mean was formed, and the relative standard deviation (standard deviation/mean) was calculated; the results are summarized in Table 2.

TABLE 2
Relative change of the power of the solar cell in the maximum
power point as a function of the incident angle of the light
beams for the above described experimental set-ups

| Incident angle | C1 | C2 | 1 | C3 | 2 |
|---|---|---|---|---|---|
| 0° | −11.5% | 38.2% | 12.7% | 9.4% | −5.1% |
| 15° | −12.8% | 50.3% | 45.5% | 16.0% | −5.0% |
| 30° | −0.6% | 56.4% | 42.5% | 19.4% | 10.9% |
| 45° | 0.1% | 79.9% | 27.2% | 15.5% | 9.4% |
| 60° | 4.7% | 84.0% | 43.3% | 48.8% | 47.4% |
| 75° | −18.5% | 140.0% | 99.5% | 76.1% | 68.1% |
| mean of 0-75° | −6.4% | 60.8% | 35.9% | 19.7% | 8.7% |

A graphical evaluation of the results obtained in Table 2 can be found in FIGS. 7a and 7b. Meanings: [symbol] black: C1; [symbol] gray: C2; [symbol] double hatching: 1; [symbol] hatching from top left to bottom right: C3; [symbol] hatching from bottom left to top right: 2.

### Discussion of the Results

The power losses in Example C1 result, on the one hand, from losses because of different refractive indices for the transitions glass/UV-curable adhesive and UV-curable adhesive/solar cell. On the other hand, part of the light beams is lost by reflection when impinging on the glass sheet. Basically, both kinds of power loss occur in all optical elements, but they are greatest with planar glass sheets.

The enhancement of the power of the solar cell in the maximum power point is predominantly determined by geometric effects, as schematically represented in FIGS. 2a/2b and FIGS. 3a/3b. The relevance of the convex geometry with faceting can be seen, above all, as the incident angle of the light beams increases. The power of the decorative element increases highly (Examples C2 and C3). For glass, this is even more significant as compared to the optical elements prepared from Pleximid® TT70.

From the wavelength-selective coating (Examples 1 and 2), losses in energy yield necessarily result, as expected, as compared to C2 and C3 because of the reflection of part of the visible spectrum. However, these can be more than compensated by the convex geometry in combination with the faceting, as seen from Table 2.

The differences between the optical elements of glass and Pleximid TT70 result from the clearly improved transmission behavior of the glass and the better surface quality of the glass specimens for manufacturing reasons.

Computer Simulation

The influence of a plano-concavo-convex geometry or plano-concave geometry of the gemstone on the power of a solar cell was examined by means of computer simulation. The simulations were performed by physical ray tracing using the program Speos of the company Optis.

### Computer Model

The CAD data of the corresponding gemstone from the measurements (see above) were used as a gemstone model. The gemstone surface was assumed to be ideal (without roughness, i.e., without surface defects). For the simulation, the refractive index of the glass of experimental Example 1 was employed, which is 1.56 at λ=550 nm.

A wavelength-selective layer or generally a boundary layer between the gemstone and solar cell was not taken into account for reasons of complexity. With the simulations, merely the influence of the different geometries (convex, concave) of the gemstone on the light yield was examined. Inclusion of the wavelength-selective layer or boundary layer would be irrelevant to this.

The solar cell was simulated with a reflecting surface with a degree of reflection of 1.3%, which is independent of the incident angle of the light. The absorption of the solar cell was 98.7%.

The irradiation of the gemstone with light was effected by analogy with the measurement in the simulation, i.e., centrally from above. The dimension of the light source was 30 mm×30 mm. The distance of the light source from the center of the seat area of the gemstone was 15 mm. The aperture angle of the light source was 2×8°. The light distribution was assumed to be Gaussian. The light source had a radiation power of 1 W. The normal light source D65 of the program Speos was used as the light source. Depending on the incident angle of the light (see below), only part of the light hits the gemstone.

### Simulations and Results

Simulation S1: Gemstone with plano-convex geometry, FIG. 8. This gemstone corresponds to the gemstone from the measurement (FIG. 5a).

Simulation S2: Gemstone with plano-convexo-concave geometry, FIG. 9. The concave recess is spherical. It is obtained by a sphere with a diameter of 18 mm. The center of the sphere lies on the normal of the area that runs through the center of the gemstone seat area. The concave recess corresponds to the ball segment with a height of 0.558 mm.

Simulation S3: Gemstone with plano-concave geometry, FIG. 10. The concave curvature of the gemstone corresponds to the convex curvature of the original gemstone, FIG. 8, inverted. The height H of the gemstone (FIG. 10) at the edges is 5 mm.

The simulations aimed at examining the influence of the geometry (concave, convex) of the optical element (gemstone) on the absorption behavior and thus on the energy yield of the solar cell as a function of the incident angle of the light beams.

The incident angle of the light beams was varied in the simulation by analogy with the measurement (see above). The absorbed radiation power in Watt of the modeled solar cell was calculated. The relative deviation of the absorbed radiation power in percent is obtained according to 100×(S2−S1)/S1 or 100×(S3−S1)/S1 and was determined at different incident angles (Table 3).

TABLE 3
Absorbed power of the solar cell as a function of
the incident angle of the light beams for the above
described simulation models, and the relative deviation
of the simulation values 100 × (S2 − S1)/S1 or 100 × (S3 − S1)/S1.

| | | | Deviation | | Deviation |
|---|---|---|---|---|---|
| Incident | | | in %: 100 × | | in %: 100 × |
| angle | S1 [mW] | S2 [mW] | (S2 − S1)/S1 | S3 [mW] | (S3 − S1)/S1 |
| 0° | 598.2 | 598.3 | 0.017 | 414.9 | −30.642 |
| 15° | 584.7 | 584.0 | −0.12 | 433.3 | −25.894 |
| 30° | 526.8 | 527.2 | 0.076 | 451.1 | −14.37 |
| 45° | 454.6 | 449.9 | −1.034 | 412.2 | −9.327 |
| 60° | 364.6 | 353.7 | −2.99 | 320.8 | −12.013 |
| 75° | 219.8 | 214.3 | −2.502 | 147.7 | −32.803 |

### Discussion of the Results

The relative deviation of the values of S3 from S1 (column 6), Table 3, shows that the absorbed radiation power strongly decreases in a purely concave geometry. This is to be expected because concave geometry has a scattering effect.

In contrast, if the surface proportion of the concave curvature is at most ⅓ (FIG. 9) of the curved area (cf. simulation 2; column 4 in Table 3), the deviation of the power values is negligible.

The relative deviations (cf. Table 3, columns 4 and 6) are not continuously decreased with respect to the incident angle. This is due to the fact that light beams also impinge on the non-curved side surfaces of the gemstone, and therefore, additional reflections may occur within the gemstone.

The simulation results show that the influence of a concave curvature having an area proportion of up to at most ⅓ of the curved gemstone surface is negligible with respect to the efficiency of a solar cell.

1. A decorative element containing
(a) a transparent gemstone with a faceted surface comprising convex curved regions,
(b) a wavelength-selective layer, and
(c) a photovoltaic cell.

2. The decorative element according to claim 1, characterized in that said gemstone is made of glass or plastic.

3. The decorative element according to claim 1, characterized in that said gemstone has a plano-convex or plano-convexo-concave geometry.

4. The decorative element according to claim 1, characterized in that said wavelength-selective layer is selected from a wavelength-selective coating or a wavelength-selective film.

5. The decorative element according to claim 4, characterized in that said wavelength-selective coating contains at least one metal and/or metal compound.

6. The decorative element according to claim 1, characterized in that said wavelength-selective layer reflects a fraction of the light within a range of from 380 to 850 nm.

7. The decorative element according to claim 1, characterized in that said wavelength-selective layer reflects at least 50% of the incident light in a 50 to 250 nm wide reflection interval within a range of from 380 to 850 nm.

8. The decorative element according to claim 7, characterized in that said wavelength-selective layer has an average transmission of >80% outside the reflection interval in a range of 400 to 1200 nm, as measured under an incident angle of the light beams of 0°.

9. The decorative element according claim 1, characterized in that said wavelength-selective layer has been applied to
(a) the side opposing the faceted side, or
(b) the photovoltaic cell.

10. The decorative element according to claim 1, characterized in that said wavelength-selective coating comprises at least one compound selected from the group consisting of Cr, Cr₂O₃, Ni, NiCr, Fe, Fe₂O₃, Al, Al₂O₃, Au, SiOₓ, Mn, Si, Si₃N₄, TiOₓ, Cu, Ag, Ti, CeF₃, MgF₂, Nb₂O₅, Ta₂O₅, SnO₂, ZnO₂, MgO, CeO₂, WO₃, Pr₂O₃, Y₂O₃, BaF₂, CaF₂, LaF₃, NdF₃, YF₃, ZrO₂, HfO₂, ZnS, Oxynitrides of Al, Si, and SnZnO, or any combination of these compounds in any sequence of layers.

11. The decorative element according to claim 1, characterized in that said photovoltaic cell is a backside-contacted solar cell.

12. The decorative element according claim 1, characterized in that components (a), (b) and (c) of the decorative element are bonded together by means of an adhesive.

13. The decorative element according to claim 12, characterized in that the refractive index of the adhesive deviates by less than ±20% from the refractive index of the gemstone.

14. An energy source for a wearable electronic device, the energy source comprising the decorative element according claim 1.

15. An object containing at least one decorative element according to claim 1.
